(19) United States
(12) Patent Application Publication
(10) Pub. No.: US 2013/0218015 A1
(43) Pub. Date: Aug. 22, 2013

(54) Method and a System for Monitoring, Contractions and/or a Birth Process and/or the Progress and/or Position of a Fetus

(71) Applicants: Yosseph Machtey, Kochav Yair (IL); Yuri Megel, Haifa (IL)

(72) Inventors: Yosseph Machtey, Kochav Yair (IL); Yuri Megel, Haifa (IL)

(21) Appl. No.: 13/613,487

(22) Filed: Sep. 13, 2012

Related U.S. Application Data
(63) Continuation of application No. 13/003,586, filed on Jan. 11, 2011, now abandoned.
(60) Provisional application No. 61/134,565, filed on Jul. 11, 2008.

(51) Int. Cl. A61B 8/02; A61B 8/06; A61B 8/00; A61B 8/08

(52) U.S. Cl. CPC A61B 8/02; A61B 8/488; A61B 8/06; A61B 8/4477; A61B 8/5223; A61B 8/54
USPC 600/454; 600/453

(57) ABSTRACT

A method of monitoring a pregnant woman by identifying a moving organ in the woman and tracking or monitoring a movement of said organ using ultrasound. Optionally, the identifying is non-imaging. Optionally or alternatively, the moving organ is part of a fetus and a position of a head is optionally calculated form a heart position which is directed detected.

### RELATED APPLICATION

The present application claims priority and the benefit under 119(e) of a U.S. provisional application Ser. No. 61/134,565 filed on 11 Jul. 2008 with title NON INVASIVE MONITOR FOR FETAL DESCENT DURING LABOR, the disclosure of which is incorporated herein by reference.

### FIELD AND BACKGROUND OF THE INVENTION

The present invention, in some embodiments thereof, relates to a system and a method for medical monitoring instrumentation and, more particularly, but not exclusively, to a system and a method for childbirth monitoring.

The degree and rate of progression of the fetus in the birth canal may be closely monitored by hospital staff during labor and childbirth, and is considered to be the cardinal indicator of the progression of labor. Inadequate descent may indicate pathological labor, and is often an indication for medical or surgical intervention, particularly if accompanied by evidence of fetal distress. Fetal descent does not ordinarily proceed at a constant rate. Moreover, fetal descent varies drastically with nulipara (women delivering for the first time) and multipara (women that have already experienced delivery).

During the course of labor several monitoring devices are routinely used such as fetal heart rate monitors, fetal oxygen saturation monitors (pulse oxymetry), uterine activity monitors (tocometry), and maternal vital signs monitors, and monitors of fetal descent. Aside from manual vaginal examination, other methods for measuring fetal descent have been described including trigonometric relative measurements of the change in position of an ultrasound or magnetic transponder located on the fetal scalp. Both of the techniques above require puncturing of the sacral sack and penetrating the fetal epidermis; either of which may cause infection. It is also commonly known that partuitents and caregivers may be reluctant to perform these procedures due to the possibility of infection.

Several systems and methods for monitoring the progress of fetal descent during labor have been developed. For example, U.S. Pat. No. 6,669,653 describes a method and apparatus for monitoring the progress of labor. The patent claims a method of monitoring the progress of labor during childbirth comprising: touching a position sensor to a point on the fetal presenting part and capturing the position of the position sensor; touching the position sensor to a set of points on the mother and capturing the position of the position sensor at each point; and monitoring the position of the point on the fetal presenting part with respect to at least one point from the set of points on the mother.

U.S. Pat. No. 6,270,458 describes a cervix dilation and labor progression monitor. Small ultrasound reflectors located on either side of the cervical os and on the fetal presenting part reflect the ultrasound signals back to extracorporeal ultrasound receivers. Ultrasound signals are analyzed to identify the relative locations of the reflectors, and the trigonometric relationships between the reflectors and transmitters are used to calculate the degree of cervical dilation, and the descent of the fetal presenting part.

U.S. Pat. No. 7,207,941 describes a medical transponder, including an ultrasonic sensor that generates electrical signals in response to impinging ultrasonic waves that it detects, an electrical connection which receives the signals, and an electromagnetic RF transmitter coupled to the electrical connection and which generates an RF signal in response to the detected waves.

### SUMMARY OF THE INVENTION

In an exemplary embodiment of the invention, there is provided a method of determining and/or tracking the position and/or head station of a fetus or particular parts thereof, by identifying the fetus using motion-generated artifacts. In an exemplary embodiment of the invention, the motion generated artifacts are generated by motion of one or more of, for example, the heart, the apex of the heart, the valves, motion of large blood vessels and/or motion of blood therein. In an exemplary embodiment of the invention, a position of a fetal head and/or descent thereof and/or change in position thereof is inferred by determining a position of a fetal heart and estimating a location and/or degree of movement of the head.

In an exemplary embodiment of the invention, the fetal heart is detected based on it including movements at an expected or measured fetal heart rate. Optionally or alternatively, the fetal heart is detected by searching for temporally near frames between which there is significant and optionally repeated movement, optionally at a substantially same distance. Optionally, such searching is by searching for optionally consecutive frames with an optionally generally high correlation and a low cross-correlation at a small part thereof, which optionally corresponds to the heart or other moving portion of interest. Optionally, the higher correlation is above a threshold or selected based on being best from a plurality of frames. Optionally or alternatively, the lower correlation is selected based on a threshold or based on relative correlation between other parts of the frames.

There is provided in accordance with an exemplary embodiment of the invention, a method for monitoring movement of a fetus in a pregnant woman comprising:

a) transmitting ultrasonic acoustic energy into the pregnant woman and the fetus;

b) receiving ultrasound acoustic energy signals modulated by a fetal moving organ;

c) analyzing the received ultrasound acoustic energy signals;

d) automatically identifying said modulation by moving organ from said analysis; and

e) estimating at least one of the location and the spatial displacement of said moving organ or said fetus based on said identifying.

Optionally, said method uses only non-imaging ultrasound. Optionally or alternatively, said estimating is in more than one dimension.

In an exemplary embodiment of the invention, said organ is cyclically moving. Optionally, said automatically identifying comprises obtain a frequency of cycles and a distance of cycling organ from an ultrasonic transducer. Optionally or alternatively, the cycling organ is the fetal heart.

In an exemplary embodiment of the invention, estimating comprises using three transducers are used together with a triangulation method to determine the location of the moving organ in space relative to the US transducers.

In an exemplary embodiment of the invention, the method comprises tracking a movement in space of said moving organ or of an organ mechanically connected to said moving organ.

In an exemplary embodiment of the invention, the method comprises:

presenting the results of said estimating.

In an exemplary embodiment of the invention, the method comprises:

monitoring a descent of said fetus based on said estimating.

In an exemplary embodiment of the invention, said presenting comprises tracking the spatial displacement of a predetermined anatomic feature of said fetus over time.

In an exemplary embodiment of the invention, said automatically identifying comprises identifying a moving anatomic feature of the fetus, based on an effect of said movement on said ultrasonic radiation. Optionally, said identifying identifies a member of a group consisting of: the heart, the valves of the heart, the apex of the heart, carotid artery blood flow and aortal blood flow.

In an exemplary embodiment of the invention, said analyzing further comprises automatically estimating a spatial displacement of a presenting part of said fetus based on an estimated distance between said presenting part and said predetermined anatomic feature, and also based on said estimating at least one of the location and the spatial displacement of said predetermined anatomic feature. Optionally, said analyzing further comprises estimating the spatial displacement of the scalp of said fetus.

In an exemplary embodiment of the invention, said analyzing comprises calculating a location of said predetermined anatomic feature by at least one of trilateration and triangulation.

In an exemplary embodiment of the invention, the method comprises connecting ultrasonic sensors to the pregnant woman for measuring the progress of labor.

In an exemplary embodiment of the invention, said transmitting acoustic energy comprises transmitting acoustic energy at a plurality of frequencies.

In an exemplary embodiment of the invention, said analysis comprises detecting a window of low correlation between consecutive frames with a high correlation.

There is provided in accordance with an exemplary embodiment of the invention, a method for determining the spatial position of the heart of a fetus in a pregnant woman comprising:

transmitting pulses of acoustic energy into the pregnant woman and the fetus at a predetermined pulse repetition frequency (PRF);

receiving echoed ultrasound acoustic energy signals originating from the transmitting;

identifying ultrasound acoustic energy signals echoed by the heart of the fetus; and

calculating the spatial position of the fetal heart based on said identifying.

In an exemplary embodiment of the invention, the method comprises:

separating the echoed ultrasound acoustic energy signals into distinct frames, said frames being characterized by a predetermined period of time;

indexing the time frame;

calculating the time of arrival (TOA) of the signals echoed by the heart of the fetus from the frames; and

calculating the fetal heartbeat rate from the frames.

There is provided in accordance with an exemplary embodiment of the invention, apparatus for monitoring descent of a fetus during childbirth in a pregnant woman comprising:

a) at least one ultrasound transmitter configured for transmitting ultrasound acoustic energy into the bodies of said pregnant woman and said fetus;

b) at least one receiver configured to receive scattered ultrasound acoustic energy signals originating from said ultrasound transmitter;

c) a controller which analyzes said signals and estimates the spatial location of a predetermined anatomic feature of said fetus therefrom.

Optionally, said controller activates an alarm to announce the onset of fetal descent. Optionally or alternatively, said at least one receiver is configured to attached to a pregnant woman extracorporeally and receives ultrasound acoustic energy signals from a probe other than itself. Optionally or alternatively, said at least one transmitter and said at least one receiver share an acoustic antenna.

There is provided in accordance with an exemplary embodiment of the invention, a method for monitoring a pregnant woman comprising:

a) transmitting ultrasonic acoustic energy into the pregnant woman and the fetus;

b) receiving ultrasound acoustic energy signals modulated by a maternal organ;

c) analyzing the received ultrasound acoustic energy signals;

d) automatically identifying said modulation by moving organ from said analysis; and

e) estimating at least one of the location and the spatial displacement of said moving organ based on said identifying.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a is a schematic block diagram of a computerized labor monitoring system for measuring head station using anatomical markers, in accordance with an exemplary embodiment of the invention;

FIG. 2 is a flowchart describing a method of monitoring fetal progress during birth, in accordance with an exemplary embodiment of the invention;

FIG. 3 is a flowchart describing a method of displaying fetal progress during birth in accordance with an exemplary embodiment of the invention;

FIG. 4A is a block diagram describing a method of initial signal processing, in accordance with some embodiments of the invention;

FIG. 4B is a block diagram describing a method of signal processing for one channel, in accordance with some embodiments of the invention;

FIG. 5 is a graph representing ultrasound echo signals from two subsequent ultrasound transmission frames, in accordance with some embodiments of the invention;

FIG. 5A is a 3D graphing of representations of ultrasound echo signals from a plurality of transmission frames, in accordance with some embodiments of the invention;

FIG. 6 is a graph representing cross-correlation between sequences of contiguous ultrasound transmission frames, in accordance with some embodiments of the invention; and

FIG. 7 is a graph representing cross-correlation between two contiguous ultrasound transmission frames, in accordance with some embodiments of the invention.

### DESCRIPTION OF SPECIFIC EMBODIMENTS OF THE INVENTION

The present invention, in some embodiments thereof, relates to a system and a method for medical monitoring instrumentation and, more particularly, but not exclusively, to a system and a method for monitoring of fetuses and/or childbirth.

The present invention, in some embodiments thereof, estimates the location and/or tracks the progression of the fetus in the birth canal by measuring the position of the heart and/or other acoustically (e.g., ultrasound) identifiable markers during birth. According to some embodiments of the present invention, ultrasound modulation based on dynamic displacement of the object is used to detect a specific anatomical marker of the fetus, for example, the heart, the carotid arteries the aorta, heart valves and/or heart apex. The relative position of the anatomical marker is then optionally monitored, and its position relative to its own previous position is the marker's relative descent. Echoed signals returned by an anatomical marker in motion are characterized by different echo signals due to Ultrasound frequency (phase) shifting or Doppler shifting. Optionally, differences in timing and/or frequency of signals received by transducers are used to create a fiducial point in three dimensional space. When the marker position is known relative to the transducers, it is used as a temporal-spatial reference point. The calculated distance between this reference point and another reference point determined at a later time represents relative fetal movement.

As used herein, the term “Doppler ultrasound” or “motion affected ultrasound” refers, without limitation, to both pulsed wave Doppler (PW) and continuous wave Doppler (CW) technologies and/or any other types of motion-affected signal processing, such as Phase shift techniques despite the different mechanisms by which by which velocity is measured. In an exemplary embodiment of the invention, CW technologies use ultrasound frequency shift to measure the velocity of an object. In an exemplary embodiment of the invention, PW technologies ignore frequency shift, but use relative phase changes of the pulses to determine a frequency shift. Typically blood-flow Doppler signals are characterized by relatively high velocities and relatively low amplitudes. Heart wall (and vessel wall) Doppler signals are characterized by relatively low velocities (4-8 centimeters/second in adults) with relatively high amplitudes. Depending on the tissue or marker sought, high frequency blood flow signals may be eliminated by gain adjustment, and myocardial echoes is optionally eliminated by a high pass filter to eliminate low amplitude signals. A potential advantage of pulsed ultrasound is the ease of obtaining TOA, which is useful for some embodiments of the invention.

In an exemplary embodiment of the present invention, when the location of the fetal heart or other motion-affected Ultrasound detectable anatomical marker has been determined, the location of the fetal head station and/or other fetal parts may be estimated. It should be noted that in some embodiments of the invention, the location of the fetal head is provided as a distance from the heart, rather than a vector. As used herein, this is termed an estimated position, however. Optionally, a user images the fetus to at least ensure that the fetus is pointing up (or down, e.g., for a breech delivery). In general, the shape of the birth canal is such that once the head enters the canal, its movement except for forward and backwards is considerably constrained and substantially any movement is optionally assumed to occur along a track defined by the birth canal.

According to some embodiments of the present invention, multiple ultrasound transducers are used to transmit and/or receive ultrasound signals. When signals are transmitted from multiple sources, a plurality of ultrasound frequencies may be used to enable one or more transducers that receive the signals to distinguish between the transmitting sources.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

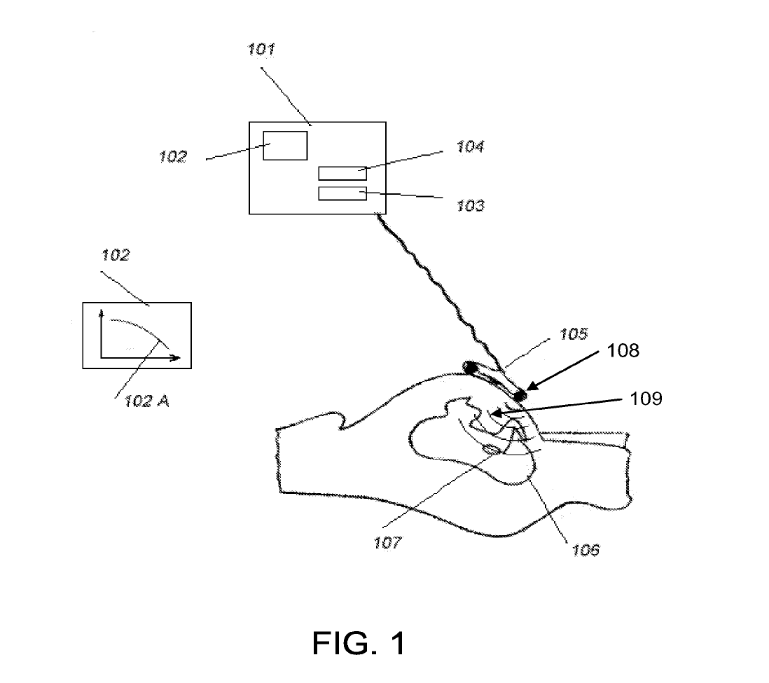

Referring now to the drawings, FIG. 1 illustrates the physical components of a device in accordance with some embodiments of the present invention. Elements of the apparatus include a control and processing unit 101 connected to (or integral with) an abdominal unit 105 comprising an extracorporeal ultrasound transmitter 108, one or more ultrasound receivers that may be comprised in the abdominal unit 105, and an optional display unit 102. Additionally or alternatively, separate external ultrasound receivers (not shown) may be used. Control and processing unit 101 instructs ultrasound transducers configured to transmit signals into a patient's body, and it receives information from ultrasound transducers configured to receive signals from the transmitting transducers. Optionally, signal information is subsequently analyzed. Abdominal unit 105 may be placed on the abdomen of the mother where it transmits and/or receives ultrasound signals. Abdominal unit 105 comprises at least one ultrasound transducer 108, and may comprise an array of transducers. Optionally, three or more transducers of the abdominal unit are arranged in a non-linear and/or triangular configuration to support a method of locating a marker, for example triangulation and trilateration. Optionally, an extracorporeal ultrasound sensor receives ultrasound signals 109 originating from ultrasound transducer 108 and/or another ultrasound transducer, details of which are sent to control and processing unit 101. Display unit 102 may receive information from control and processing unit 101 to present information 102A to users of the device.

In an exemplary embodiment of the invention, control and processing unit 101 is a general purpose computer, for example, a personal computer. Alternatively, control and processing unit 101 is a microcontroller that controls transmission and/or reception of acoustic signals. Optionally, control and processing unit 101 comprises a pulse generator, a data acquisition system, and/or a display unit. Optionally, unit 101 includes a user input, for example, for a user to enter settings for transmission, processing and/or display.

According to some embodiments of the present invention, a vaginal probe (not shown) operates as an ultrasound transmitter and/or receiver. This embodiment is similar to the embodiment described immediately above; however, ultrasound signals are transmitted and/or received by the probe inside of the patient's body. Optionally, the vaginal probe may be used in conjunction with abdominal unit 105. Alternatively, the vaginal probe may work independently and/or in the absence of abdominal unit 105.

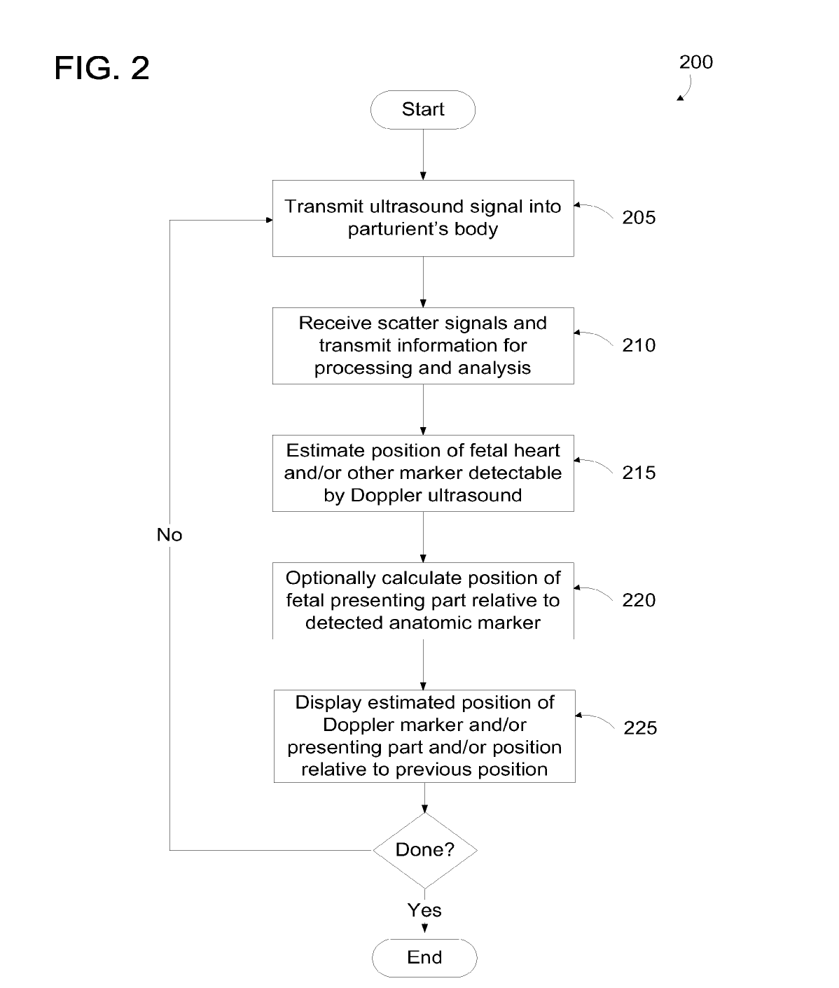

Reference is now made to FIG. 2, which is a flowchart describing a method 200 of monitoring fetal progress during birth, in accordance with an some embodiments of the invention.

At least one ultrasonic transducer 108 transmits an ultrasound acoustic signal 109 into a parturient's body as an ultrasound pulse (205). The acoustic signal 109 travels through the body, scatters from interfaces between tissues with different mechanical properties. The scattered signals are detected by at least one ultrasound transducer and transmitted to control and processing unit 101 (210). By implementing motion detection techniques, for example, Doppler frequency change or sound wave phase shift, anatomical structure and/or blood flow movement are also detected. Scattered ultrasound signals from the fetal heart 107, aorta, and/or carotid artery may be detected, and one or more of those objects may be used as markers for fetal movement and/or position and/or orientation (e.g., if two spatially separated markers are used).

In an exemplary embodiment of the invention, the fetal heart is particularly detected based on an estimation of a range of fetal heart beat rate (which is generally different and higher from maternal heart beat) and/or based on an input from a fetal ECG or heart rate sensor. Optionally, the fetal heart rate is detected using the ultrasonic signal, for example, by scanning the range covered by the transducers and analyzing for possible FHR data (e.g., at different distances from the transducers).

Optionally, signal processing and analysis (215), performed by control and processing unit 101 determines the spatial position of a fetal marker detectable by Doppler, for example, the fetal heart, by methods known in the art.

Optionally, digital processing techniques (e.g., frequency filtering, depth gating) isolate and/or remove unwanted echoes from body structures located between a transducer and a region of interest, for example, the fetal marker. Optionally or alternatively, such processing techniques are used to isolate signals indicative of motion.

The spatial position of the presenting part, usually the fetal scalp, may be estimated (220) using distances between anatomical structures. Medical staff may perform this estimation. Optionally, the values of those distances may be used to calibrate the device. Displacement of the fetal scalp due to natural movement, for example, head rotation unrelated to the birth process is optionally not detected by method 200, however, this type of movement is not a significant indication of fetal descent. Optionally, display unit 102 presents estimated positions of a fetal marker and/or the presenting part, relative to their previously estimated positions 225. Additionally or alternatively, display unit 102 displays optional information, for example, extent of cervical dilation, vital signs, and/or indicators of tocolysis, or information in accordance with, for example, the teaching of PCT publication WO2005/096707. It should be noted that in some embodiments of the invention, the location of the head is not estimated and/or known, rather, what may be of interest is the movement of the fetal body (e.g., the heart), for example, over time and/or in response to contractions of the uterus.

It is noted that, in general, the birth canal is 10 cm long, hence the fetal scalp advances about 10 cm from the onset of birth until delivery. Some embodiments of the invention are based on the assumption that measuring the displacement of the heart yields similar results to the measurement of the displacement of the fetal scalp. Though other monitoring devices may be somewhat more accurate since they measure the displacement of the fetal scalp directly, in pathological states such as molding and caput, other such other devices would possess an inherent progressive or fixed inaccuracy while the heart's displacement does not posses such an inaccuracy. It should be noted that in some embodiments of the invention, the heart does not serve as a proxy for the scalp. Rather, location and/or movement of the heart are used to provide information re the movement of the body of the fetus. If two movement-markers are tracked on the fetus, an orientation in space, or a movement vector, may be reconstructed. In one example, a user identifies the two markers, for example using imaging (if imaging is used, which it is optionally not) or based on the signals. Optionally or alternatively, the system shows a window or where a second marker (e.g., brain vessels) may be, base don detection of the heart, or may calculate such a window for automatic detection of a second marker, based on anatomical considerations and/or user input. This may be done, for example, using PW or CW. In an exemplary embodiment of the invention, blood vessels of the fetus are identified by them pulsing at the heart rate (and optionally in synchronization with at a fixed delay from the heart.

There are several methods known in the art for processing signal information based on the non-stationary character of a detected object, e.g., a heart. For example, see Mundigler G., Zehetgruber M., “Tissue Doppler Imaging: Myocardial Velocities and Strain—Are there Clinical Applications?”; Journal of Clinical and Basic Cardiology 2002; 5 (Issue 2):125-32. Doppler frequency shift measurements in the signal scattered from a moving object (e.g., blood, walls of vessels, tissues) are widely used in tissue Doppler imaging, for example, in detecting blood flow and measuring strain rate.

Another method to detect a moving object is to exploit the shift of an echo signal in the time-domain window, for example, using PW ultrasound. Combined analysis of two subsequent frames (i.e., echoes from two ultrasound pulses) by cross correlation functions allow determination of where the windowed data of the one echo matches the second echo and is shifted in time due to object motion. The time interval between the onset of the acoustic pulse and the detection of its echo signals is used to calculate distances between the transducers (which may be placed on the patient's abdomen) and a fetal heart. Both methods allow determination of the region on the axis of the beam where the moving object is present and reduces the signals scattered from stationary structures in the body. The spatial location of the fetal heart may now be calculated by methods such as triangulation or trilateration. As will be clarified below, in some cases, such precise/multi-dimensional determination is not needed.

In some embodiments, abdominal unit 105 comprises at least one extracorporeal ultrasound transmitter that further comprises at least three ultrasonic transducers 108, optionally arranged to be triangularly situated. Three or more non-linearly situated ultrasonic transducers that transmit signals 109 at distinct frequencies (described below) and/or three or more non-linearly situated ultrasonic transducers that receive transmitted signals may be used to locate an object by methods including trilateration (presently described) or triangulation. Optionally, the transducers provide some angular indication, for example, acting as phased arrays or including some angular-dependent signal property.

Trilateration is a method by which distances between an object and each of three different sensors (not aligned on a single line) are used to isolate the location of the object to two possible points (or in some instances to one point). The two possible points can be further reduced to one point either by obtaining the object's distance to a fourth sensor or by algorithmically providing additional constraints. If the three sensors are ultrasound transducers adjacent to the abdomen of a patient, the plane defined by the three points (sensors) is substantially tangent to the surface of the abdomen, therefore, one of the two solution points will lie under the sensors, i.e., in the patient's body, and the second solution point will lie above the sensors, i.e., outside of the patient's body and readily eliminated. Using this method, the spatial position of a fetal anatomical marker detectable by ultrasound can be determined. The set of all points equidistant from a single point defines the surface of a sphere. The intersection of two such spheres defines a circle. The intersection of that circle with a third such sphere defines two points, or in the case where the circle and surface of the third sphere are tangent, a single point. Optionally, while there is some ambiguity in the intersection, it is assumed that the fetus is on one side of the transducers, so any solution outside the body is ignored.

Optionally, two or more sensors configured to detect the spatial angular direction of received ultrasound signals are used to detect the location of a fetal marker by means of triangulation. It should be noted that the accuracy of the determined location can be, for example, 2 cm, 1 cm or 0.5, depending on the embodiment if the invention.

Optionally, one sensor configured to detect the spatial angular direction of received ultrasound signals and two or more sensors possibly not configured to detect the spatial angular direction of received ultrasound signals are used to calculate the location of a fetal marker algorithmically.

Optionally, the three or more ultrasound transducers may each transmit ultrasound signals at a different and distinct frequency so that the one or more sensors can distinguish between echo signals originating from each transmitting transducer. Marker location is optionally determined by methods described herein.

Optionally, the calculation of a fetal marker by triangulation is performed as described in U.S. Pat. No. 6,270,458, the disclosure of which is which is incorporated herein by reference.

Optionally, the system calculates a Boolean value indicating that a fetal marker has moved a predefined distance from a baseline and/or is present or absent in a predefined portion of space and/or has passed a threshold location. This may be used to determine a phase of childbirth. For example, this method may be used to detect that the head of the fetus is wholly in the birth canal, based on the heart or scalp having passed an imaginary line in space. Optionally, such a line is defined using an appropriately located transducer. Optionally, a vaginal transducer is used for such detection.

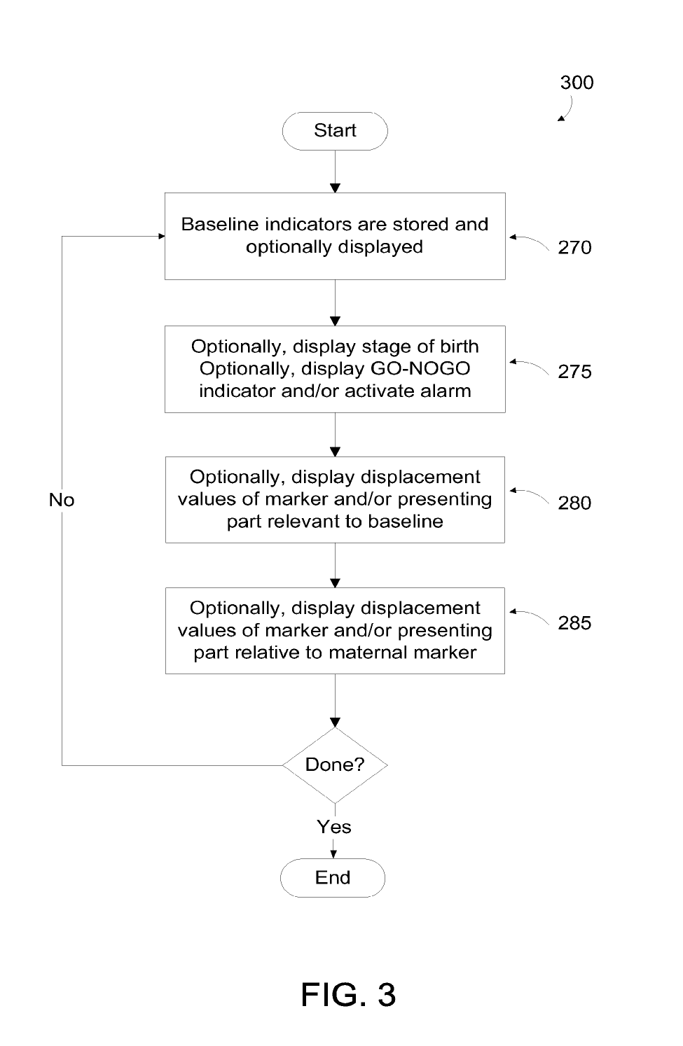

Reference is now also made to FIG. 3 which is a flowchart describing a method of displaying fetal progress during birth, in accordance with some embodiments of the invention. Baseline measurements of a fetal marker and/or a presenting part are optionally stored, e.g., by a control and processing unit 101 for subsequent retrieval and optionally displayed (270) on an optional display 102 (e.g., or sent to a remote location for processing and/or display). For example, an initial location of a fetal marker such as a heart 107 and/or a scalp 106 may be presented.

Optionally, a Boolean indicator is calculated and displayed to indicate that a predetermined stage of birth is reached (275). Optionally, a GO-NOGO indicator may be presented. For example, when a 2 centimeter displacement of a fetal marker relative to its baseline value is calculated, the indicator may be switched on. Optionally or alternatively, when the Boolean indicator is set, the control and processing unit 101 activates an alarm that is displayed and/or sounded.

A calculated relative displacement of a fetal marker and/or presenting part may be displayed, optionally with a history of its baseline value and previously estimated relative displacements (280).

Optionally, when a marker connected to part of the body of the pregnant woman, for example, the cervix is also identified and its location estimated, a displacement of a fetal marker and/or presenting part may be displayed relative to the maternal marker (285).

The process is optionally repeated, until birth is complete or until the medical staff decides to end it. In an exemplary embodiment of the invention, the data is sampled at a rate of 1 second, or at other rates, for example, between 10 times a second and once in 10 or 20 minutes, for example, once in 20 seconds, once a minute or intermediate rates. In an exemplary embodiment of the invention, note is taken of the effect of contractions on the measurements, e.g., on the movement of the fetus advancement during contraction and/or retraction after contraction. Optionally, the sampling rate at such times is made higher.

Information may be displayed on a customized screen configured to operate with the current invention and/or on a display device associated with a general purpose computer. Optionally or alternatively, information may be recorded on paper and/or other hardcopy media.

Optionally or alternatively, tracking information may be stored for subsequent retrieval on magnetic and/or electronic media. Optionally or alternatively, a time dimension is displayed, for example, a display monitor may show a graph of linear displacement of the fetal heart at time intervals and/or marker displacement may be recorded on a continuous paper roll that progresses at a fixed rate, for example, 1 cm per minute.

In some embodiments of the present invention, a monitor is used to track linear displacement of a fetal marker in a single dimension. An operator may set a relative displacement threshold for the marker beyond which an alarm to signal the onset of late stages of parturition is activated

Optionally, the device may calculate and/or present statistical transformations of data, for example, time-averaged linear displacement of a marker. This may be used to smooth short term displacements, e.g., as caused by caused by maternal contractions, fetal motion, and/or other transient factors affecting marker displacement. Such statistical transformations may provide more reliable indicators of birth progress than raw measurements.

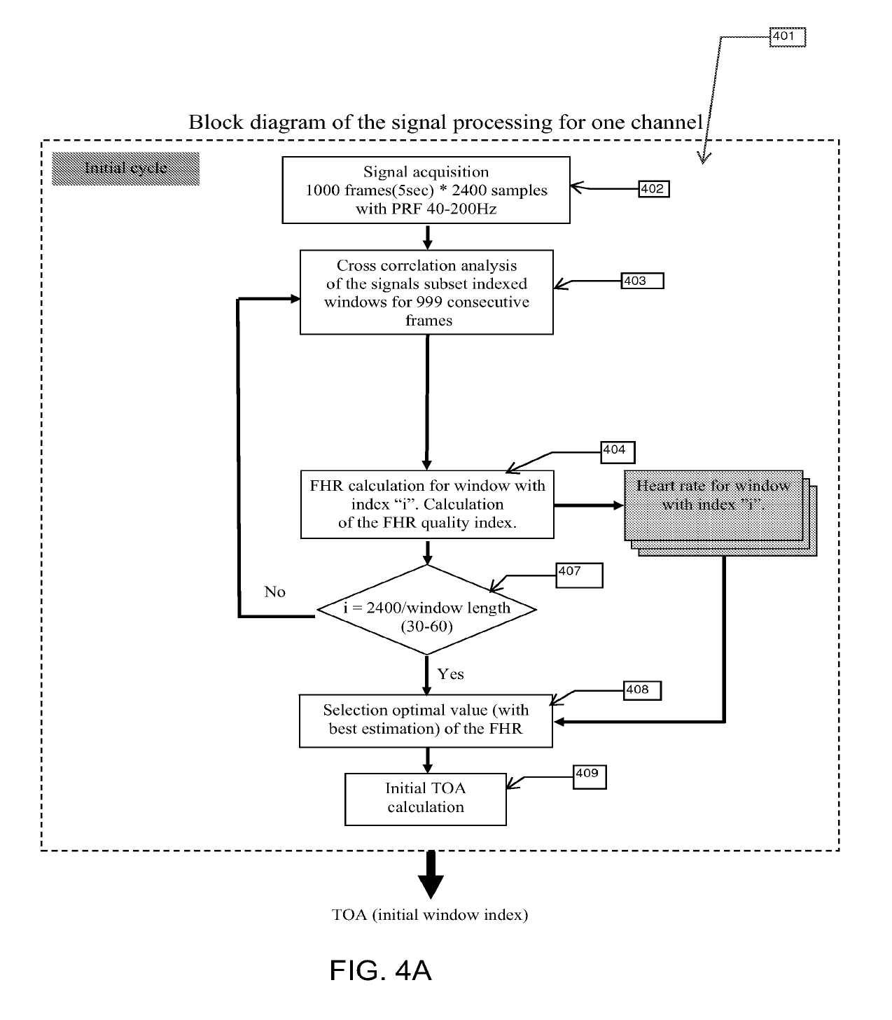

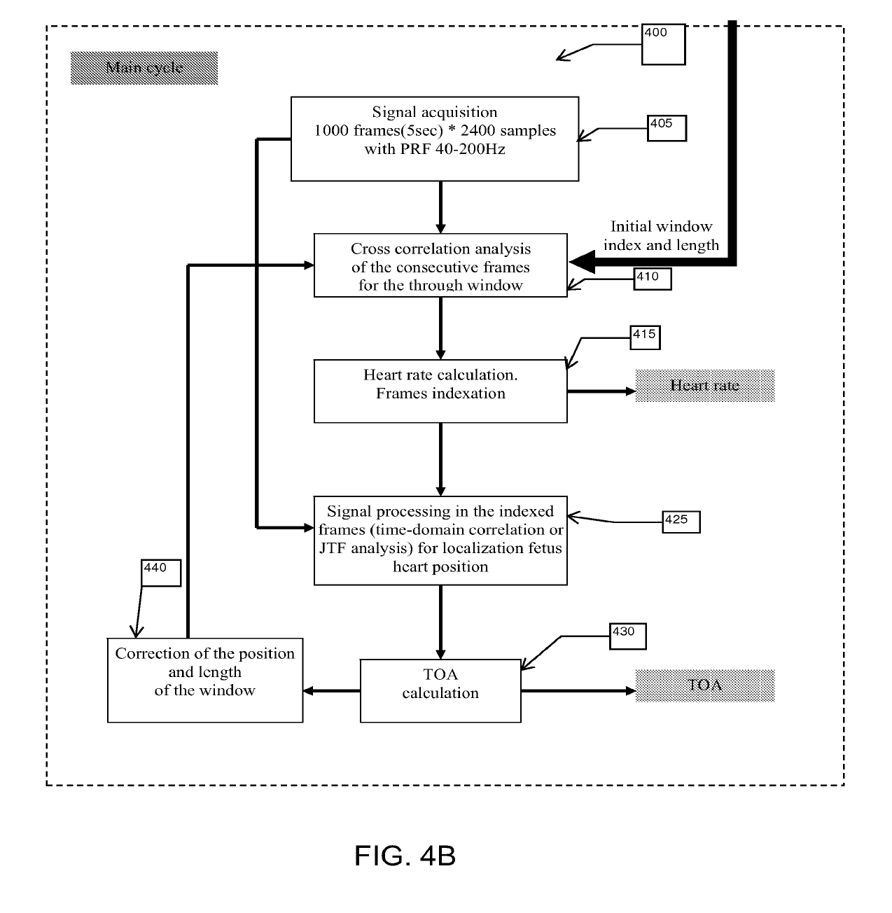

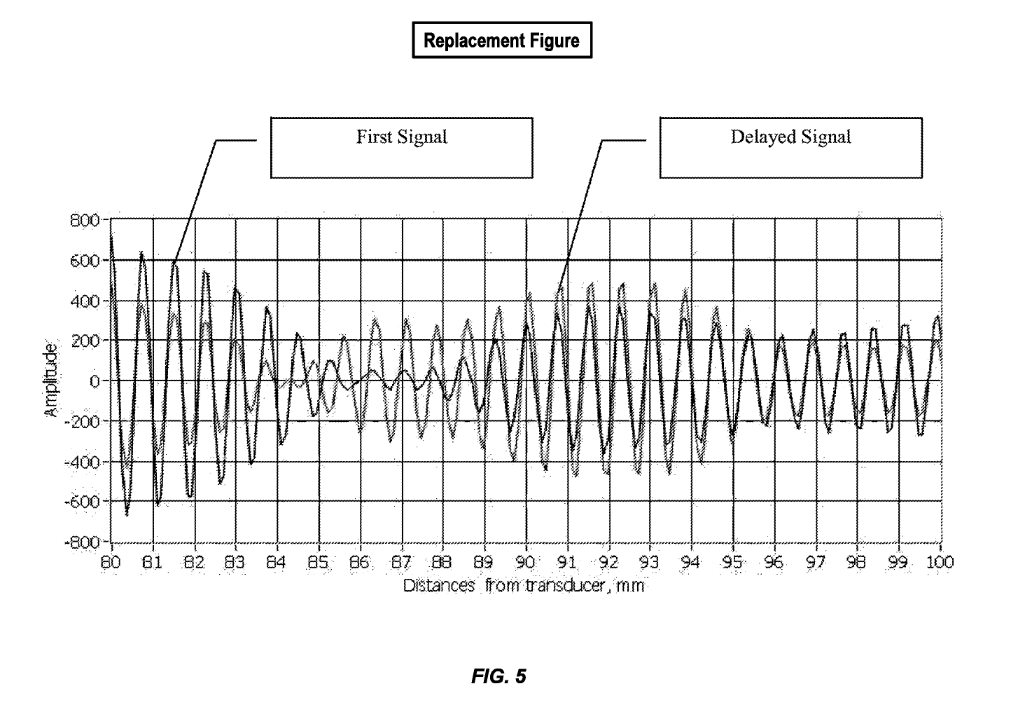

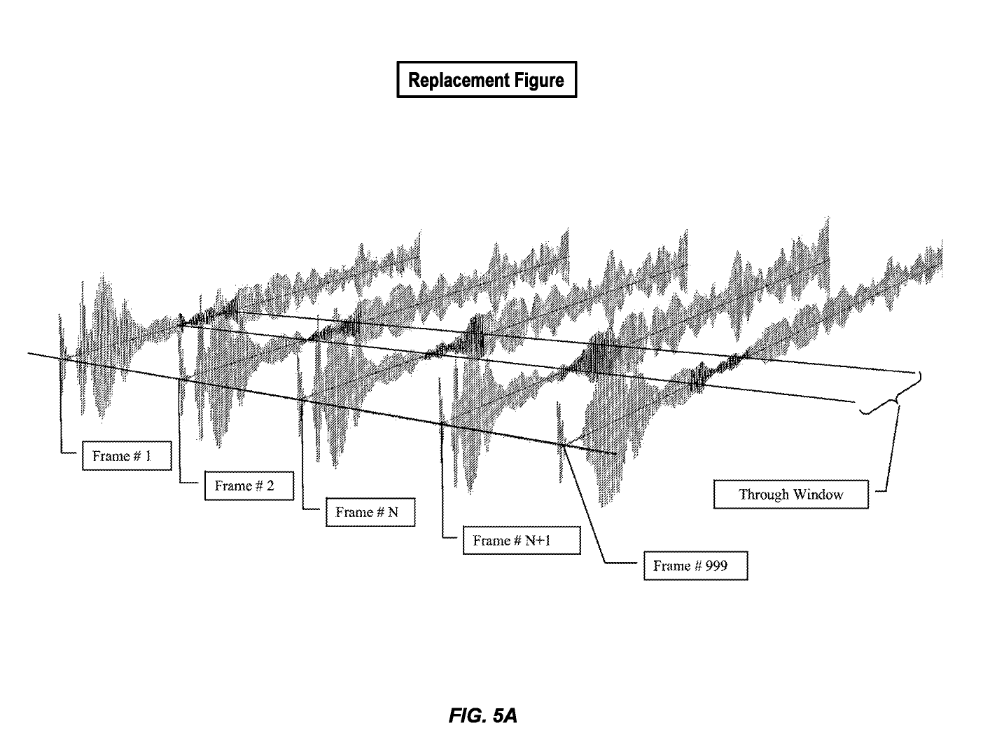

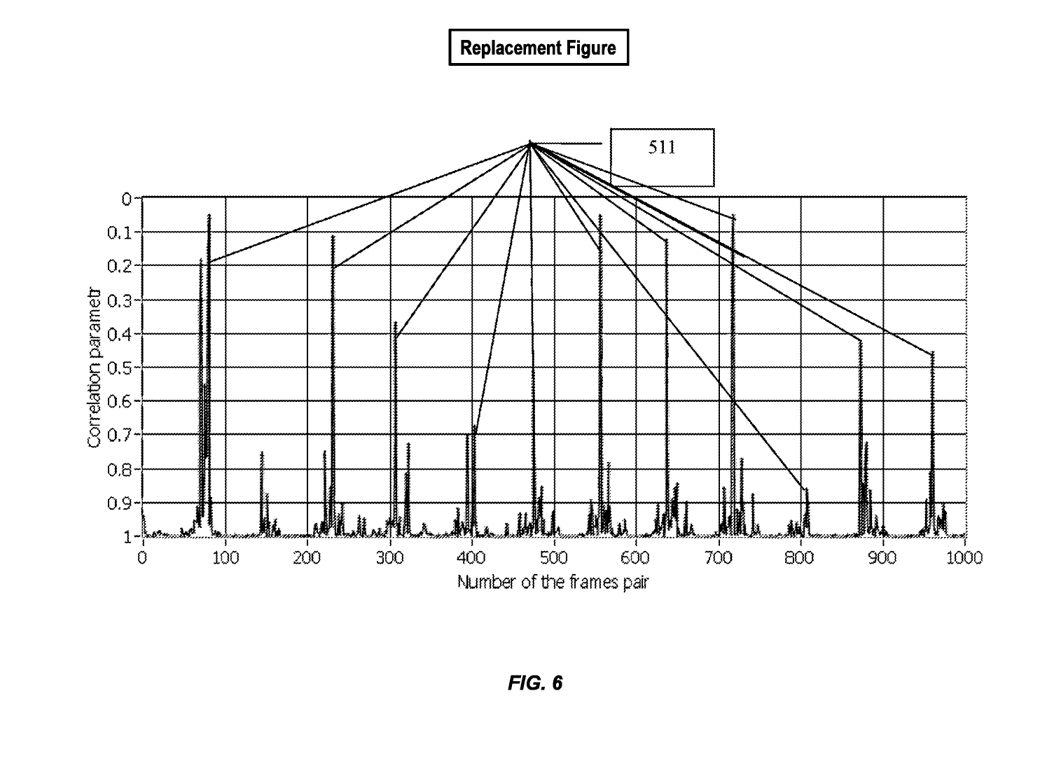

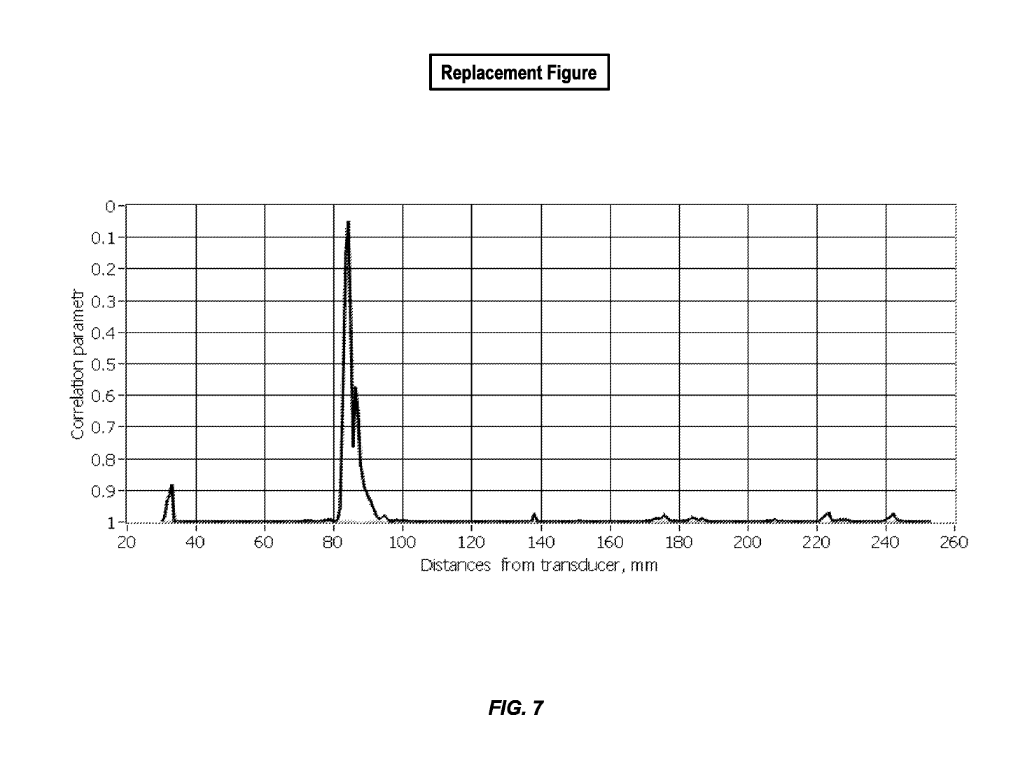

In an exemplary embodiment of the invention, the system first detects the fetal heart rate and then uses that detection to assist in detecting the marker. Optionally, this process is repeated, for example, periodically or if there is a loss of signal and/or if a transducer is determined to have moved and/or based on movement of the fetus (e.g., out of a previously determined window. It is noted that during most of the birth process the heart is not blocked form the abdominal transducers by the pelvic bones. In the following description of an exemplary signal processing method, FIG. 4A shows an initial processing, FIG. 4B shows ongoing processing, FIG. 5 shows two overlapped signals, FIG. 5A shows a plurality of frames side by side and FIGS. 6 and 7 illustrate further processed signals.

In an exemplary embodiment of the invention, the initial cycle includes scanning the entire abdomen or range of the transducers to look for potential heart signals. This may be done, for example, with one transducer, with each transducer or with all of them together. In an exemplary embodiment of the invention, once heart rate is determined for one transducer it is used for all transducers. Optionally or alternatively, it is detected in several transducers and the best one is used or a heart rate is estimated form the input of two or more transducers. Optionally or alternatively, a different time slot may be assigned for each transducer to be used. Such selective using of one or more transducers may be applied during ongoing process.

In an exemplary embodiment of the invention, initially, the window within which the heart is searched for is large, e.g., approximately 100 mm or more, and is located approximately 50-200 mm from the transducers. The window is subsequently moved and reduced in size (e.g., to 20-100 mm or about 50 mm), e.g., using adaptive techniques.

FIG. 4A is a flowchart 401 of a method of optional initial setting of a window for analysis, in accordance with an exemplary embodiment of the invention. Optionally or alternatively, an initial setting may be guessed or provided by a user.

At 402, signals are acquired (e.g., form each transducer separately), for example, 1000 frames (e.g., ˜5 seconds) at 2400 samples each, for example, with a PRF of 40-200 Hz. Other numbers of frames, samples and frequencies may be used as well, for example, between 200 and 400 frames, between 200 and 5000 samples and/or between 10 and 1000 Hz.

At 403, a cross-correlation of the frames (or of one frame with others of the frames) is calculated.

At 404, a fetal heart rate is calculated for a window with an index i. Optionally, the quality of the FHR determination, is estimated. More details are described below.

At 407, a check is made if the index should be updated, if so, the cross-correlation is repeated with a new index window.

At 408, a best FHR is selected form those calculated, for example, based on its quality and/or its reasonableness (e.g., known fetal heart rates, or input form an ECG).

At 409, an initial time of arrival calculation is made to estimate toe time of arrival at each transducer. This initial position estimate and the window estimate are then used for ongoing calculations (FIG. 4B).

Referring to Fig. to 4B, which is a flowchart 400 of a signal processing for one channel, in accordance with some embodiments of the invention. At 405, a PW ultrasound signal is transmitted and received by a transducer, consists of for example, 600 to 1000 short burst transmissions designated as frames. A frame rate is optionally configured at 200 Hz (3 to 5 seconds), providing data from 2400 to 3072 samples during four to six cardiac cycles. Each frame contains signal samples typically scattered from distances up to 20 cm (e.g., between 10 and 30 cm, optionally based on the window of FIG. 4A and/or on user input).

At 410, frames, optionally consecutive frames are cross-correlated. Optionally, the position (index) of the window in a frame is obtained from the initial cycle (e.g., estimated heart position) or from previous ongoing cycle.

At 415, FHR is calculated, for example, as described below. Optionally or alternatively, frames are indexed

At 425, the indexed frames are processed to locate the heart (e.g., for each transducer).

At 430, time of arrival is calculated, e.g., for all transducers.

At 440, the position and/or length of the window are optionally updated.

Reference is now also made to FIG. 5, which is a graph representing ultrasound echo signals from two subsequent frames obtained with delay of 5 milliseconds, in accordance with some embodiments of the invention. As seen in FIG. 5

The signals from two sequential frames can differ one or both of their phase shift and frequency shift. It is believed that high correlation of two sequential frames is an indication that the heart wall did not substantially move between times that the frames were obtained. Possibly, the maximum of a cross-correlation function occurs when the signals of the frame have a high degree of similarity between them. It is expected that this occurs between heartbeats (e.g., atrial systole, isovolumetric relaxation) when the heart is relatively motionless; possibly correlation is high because signals a few milliseconds apart, scattered by stationary objects, are similar. However, signals from frames during active parts of the cardiac cycle (rapid ejection and rapid ventricular filling) possibly exhibit lower cross-correlation function values.

Referring now to FIG. 5A, Following calculation of a cross-correlation function for all adjacent frames (1-2, 2-3 . . . ) a single parameter (such as maximum, leg of maximum or the value at “0” leg) is extracted. Cyclic characteristic of the motion can be revealed once the parameter is plotted as a function of time. (FIG. 6).

The graph of FIG. 6, represents the result of the cross-correlation analysis of the 999 pairs of frames in the present example, and shows the periods of heart wall motion at the localized maxima 511, i.e., at points of low frame-pair correlation. The obtained time sequences with cyclic character is used for FHR calculation. In some embodiments, no actual plotting is carried out, rather the equivalent mathematical procedures are applied (e.g., extracting cyclic character).

Referring back to FIG. 5A, to increase the detection sensitivity a smaller temporal window is optionally employed. The location of the required window in the long signal frame is optionally determined based on the best result of the FHR sequence in which a repeatable signal is obtained.

In an exemplary embodiment of the invention, the FHR is obtained from FIG. 6 using Frequency Domain Techniques such as FT or Priodograms.

Thereafter a more exact distance to the heart is optionally determined.

Referring to FIG. 6, the complexes with the largest peak (e.g., lowest correlation) are selected. Optionally, to eliminate artifacts only peaks with a steady HR are evaluated.

In one embodiment, when FHR is calculated, frames of minimal cross-correlation (e.g., high charge of heart geometry) are indexed, and time domain analysis is performed to determine the heart's position. Such a technique is described in Foster, S. G. Embree, P. M. O'Brien, W. D., Jr., “Flow Velocity Profile via Time-Domain Correlation: Error Analysis and Computer Simulation”, IEEE Transactions on Ultrasonics, Ferroelectrics and Frequency Control, May 1990, Volume: 37, Issue: 3, pages: 164-175, ISSN: 0885-3010. For the analysis of two consecutive frames, Fn(t) and Fn+1(t), where n is the time frame index and t is the time within the frame, the cross correlation function between two windowed parts of the frame is defined by

$$R ( τ ) = {\int }_{{T}_{1}}^{{T}_{2}} {F}_{n} ( t ) {F}_{n + 1} ( t ) d t ;$$

where T1 and T2 are start and end points of the window, respectively. The sensitivity of the function to heart movement can become greater as the window becomes smaller in the vicinity of the heart echo. Reference is now also made to FIG. 7, which is a graph represents the result of the cross-correlation analysis between two contiguous ultrasound transmission frames associated with the selected peak in FIG. 6. It should be noted that an analysis of more than two frames and/or non-consecutive frames, maybe undertaken. When the heart echo is determined, the distance between the transducer and the heart, i.e., time of arrival (TOA), is calculated (e.g., 430), for example, based on the location of the most significant peak. The time is used to correct and/or fine-tune the window that is used in the next pair of frames (e.g., 440). Similarly, TOA is optionally obtained in three separate channels, allowing triangulation calculation of the spatial position of the heart as described in U.S. Pat. No. 6,270,458.

According to some embodiments of the present invention, one or more ultrasound reflectors and/or passive ultrasound sensors may be placed adjacent to the cervix and/or attached to the fetal scalp 106. These reflectors and/or sensors may serve to identify the degree of cervical dilation. (See, for example, U.S. Pat. No. 6,270,458.) Used in conjunction with the identification of a fetal marker by a method described above, reflector and/or sensor positions may be correlated to the location of a fetal marker (e.g., heart), to measure the progress of labor, for example, by estimating the position of the fetal marker and/or a presenting part relative to the cervix and/or for calibrating. It should be noted that unlike some other embodiments heretofore described, this embodiment is somewhat invasive and may comprise attendant complications. Optionally or alternatively, internal sensors are used in conjunction with external sensors to estimate fetal marker position by triangulation, trilateration and/or another method. Optionally or alternatively, the position of a reflector and/or sensor attached to the fetal scalp 106 is correlated to the location of a fetal marker (e.g., heart), to detect fetal birth trauma, for example, caput succedaneum. Changing distances over time between a fetal marker and a scalp sensor may indicate such trauma.

In an exemplary embodiment of the invention, optional calibration, optionally periodic, is provided by vaginaly inserting a probe having a marker (e.g., reflector or vibrator with known frequency) on its tip, said probe optionally being inserted until it contacts a fetal head. Once inserted, the above described system can simultaneously determine the location of the physiological marker and of the probe marker and calculate a distance there between. Optionally, an imaging system is used for such calibration and/or for estimating a distance between a fetal head and a heart (or other landmark) and/or for detecting a change in such value due to bending of the fetal neck and/or skull elongation. It is noted, however, that the methods described above, including calibration may be carried out using a non-imaging system.

According to some embodiments of the present invention, one or more wireless ultrasound sensors inside and/or outside the body receive ultrasound signals and convert them to radiofrequency (RF) signals for transmission. The sensors transmit the RF signals to one or more RF receivers connected to a control and processing unit. Optionally, the sensors transmit the RF signals at a plurality of RF frequencies, thus allowing the RF receivers to distinguish between different transmitting sensors.

Optionally, the system is used as a stand-alone unit. Alternatively, the system is incorporated into another system that comprises additional monitoring functions, for example, cervical dilation and fetal heart rate.

In some embodiments, the above methods are used to track movements of other body parts, for example, the maternal uterus, for example, by selecting an appropriate distance window thereto (e.g., for near side) and then optionally searching for a body part with correlated movement at a significantly displaced window. Such tracking can be, for example at a same time as tracking fetal movement or instead of.

In an exemplary embodiment of the invention, a display is provided showing one or more of fetal movement (e.g., position and/or vector), contractions, fetal heart rate (e.g., extracted using the above methods) and/or other birth related and/or pregnancy related data.

It should be noted that the above methods and system may also be used before the birth process starts, for example, as part of maternal prenatal monitoring.

It is expected that during the life of a patent maturing from this application many relevant fetal position monitors will be developed, and the scope of the term fetal position monitor is intended to include all such new technologies a priori.

As used herein the term “about” refers to ±10.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for monitoring movement of a fetus in a pregnant woman comprising:
a) transmitting ultrasonic acoustic energy into the pregnant woman and the fetus;
b) receiving ultrasound acoustic energy signals modulated by a fetal moving organ;
c) analyzing the received ultrasound acoustic energy signals;
d) automatically identifying said modulation by moving organ from said analysis; and
e) estimating at least one of the location and the spatial displacement of said moving organ or said fetus based on said identifying.

2. A method according to claim 1, wherein said method uses only non-imaging ultrasound.

3. A method according to claim 1, wherein said estimating is in more than one dimension.

4. A method according to claim 1, wherein said organ is cyclically moving.

5. A method according to claim 4, wherein said automatically identifying comprises obtain a frequency of cycles and a distance of cycling organ from an ultrasonic transducer.

6. A method according to claim 4, where the cycling organ is the fetal heart.

7. A method according to claim 1, wherein estimating comprises using three transducers are used together with a triangulation method to determine the location of the moving organ in space relative to the ultrasonic transducers.

8. A method according to claim 1, comprising tracking a movement in space of said moving organ or of an organ mechanically connected to said moving organ.

9. A method according to claim 1, comprising:
presenting the results of said estimating.

10. A method according to claim 1, comprising:
monitoring a descent of said fetus based on said estimating.

11. The method of claim 9, wherein said presenting comprises tracking the spatial displacement of a predetermined anatomic feature of said fetus over time.

12. The method according to claim 1, wherein said automatically identifying comprises identifying a moving anatomic feature of the fetus, based on an effect of said movement on said ultrasonic radiation.

13. The method of claim 12, wherein said identifying identifies a member of a group consisting of: the heart, the valves of the heart, the apex of the heart, carotid artery blood flow and aortal blood flow.

14. The method according to claim 1, wherein said analyzing further comprises automatically estimating a spatial displacement of a presenting part of said fetus based on an estimated distance between said presenting part and said predetermined anatomic feature, and also based on said estimating at least one of the location and the spatial displacement of said predetermined anatomic feature.

15. The method according to claim 1, wherein said analyzing further comprises estimating the spatial displacement of the scalp of said fetus.

16. The method according to claim 14, wherein said analyzing comprises calculating a location of said predetermined anatomic feature by at least one of trilateration and triangulation.

17. The method according to claim 1, further comprising connecting ultrasonic sensors to the pregnant woman for measuring the progress of labor.

18. The method according to claim 1, wherein said transmitting acoustic energy comprises transmitting acoustic energy at a plurality of frequencies.

19. A method according to claim 1, wherein said analysis comprises detecting a window of low correlation between consecutive frames with a high correlation.

20. A method for determining the spatial position of the heart of a fetus in a pregnant woman comprising:
transmitting pulses of acoustic energy into the pregnant woman and the fetus at a predetermined pulse repetition frequency (PRF);
receiving echoed ultrasound acoustic energy signals originating from the transmitting;
identifying ultrasound acoustic energy signals echoed by the heart of the fetus; and
calculating the spatial position of the fetal heart based on said identifying.

21. The method of claim 20, further comprising:
separating the echoed ultrasound acoustic energy signals into distinct frames, said frames being characterized by a predetermined period of time;
indexing the time frame;
calculating the time of arrival (TOA) of the signals echoed by the heart of the fetus from the frames; and
calculating the fetal heartbeat rate from the frames.

22. An apparatus for monitoring descent of a fetus during childbirth in a pregnant woman comprising:
a) at least one ultrasound transmitter configured for transmitting ultrasound acoustic energy into the bodies of said pregnant woman and said fetus;
b) at least one receiver configured to receive scattered ultrasound acoustic energy signals originating from said ultrasound transmitter;
c) a controller which analyzes said signals and estimates the spatial location of a predetermined anatomic feature of said fetus therefrom.

23. The apparatus of claim 22, wherein said controller activates an alarm to announce the onset of fetal descent.

24. The apparatus according to claim 22, wherein said at least one receiver is configured to attached to a pregnant woman extracorporeally and receives ultrasound acoustic energy signals from a probe other than itself.

25. The apparatus according to claim 22, wherein said at least one transmitter and said at least one receiver share an acoustic antenna.

26. A method for monitoring a pregnant woman comprising:
a) transmitting ultrasonic acoustic energy into the pregnant woman and the fetus;
b) receiving ultrasound acoustic energy signals modulated by a maternal organ;
c) analyzing the received ultrasound acoustic energy signals;
d) automatically identifying said modulation by moving organ from said analysis; and
e) estimating at least one of the location and the spatial displacement of said moving organ based on said identifying.
